(19) United States
(12) Patent Application Publication
(10) Pub. No.: US 2011/0295174 A1
(43) Pub. Date: Dec. 1, 2011

(54) BANDAGE WITH BUILT-IN RAISER

(76) Inventor: Brad Richards, Morgan Hill, CA (US)

(21) Appl. No.: 13/118,694

(22) Filed: May 31, 2011

Related U.S. Application Data
(60) Provisional application No. 61/349,618, filed on May 28, 2010.

(51) Int. Cl. A61F 13/02

(52) U.S. Cl. 602/54

(57) ABSTRACT

A bandage with built-in raiser includes a flexible material having a lower side; first and second flat portions of the lower side of the material having an adhesive surface; a raised portion of the lower side of the material having a non-adhesive surface; and a raiser that transitions the material from the level of the first flat portion to the level of the raised portion and then back to the level of the second flat portion, so that the non-adhesive surface is raised relative to the adhesive surface. The non-adhesive surface may include a layer of gauze, and may be raised so as to pass air between a wound and the bandage when the bandage is applied to a user.

### RELATED APPLICATIONS

This application claims the benefit of the filing date of U.S. patent application Ser. No. 61/349,618, filed May 28, 2011, which is incorporated herein by reference in its entirety.

### BACKGROUND OF THE INVENTION

The present invention generally relates to bandages and more specifically to bandage with a built-in raiser.

Current badges have a strip of flexible material with an adhesive layer at the ends and gauze in the center. The gauze extends out, away from the strip, and may press upon a user's wound, trapping air and causing discomfort.

It would be desirable to have a bandage that raises the gauze away from the wound.

### SUMMARY OF THE INVENTION

In one aspect of the present invention, a bandage includes a flexible material having a lower side; first and second flat portions of the lower side of the material having an adhesive surface; a raised portion of the lower side of the material having a non-adhesive surface; and a raiser that transitions the material from the level of the first flat portion to the level of the raised portion and then back to the level of the second flat portion, so that the non-adhesive surface is raised relative to the adhesive surface.

In another aspect of the present invention, a bandage includes a conforming member forming one side of the bandage, the conforming member having two opposite ends and a central part; a pressure sensitive adhesive layer on the two opposite ends; and a non-adhesive raised surface within the central part, the non-adhesive raised surface adapted to pass air between a wound and the bandage when the bandage is applied to a user.

In yet another aspect of the present invention, a raiser for a bandage having adhesive portions and a non-adhesive portion includes lower sections, for attaching to the adhesive portions of the bandage; an upper section, for attaching to the non-adhesive portion of the bandage; and transition sections that retain the upper section back from the lower sections so that the non-adhesive portion of the bandage is raised relative to the adhesive portions of the bandage.

### BRIEF DESCRIPTION OF THE DRAWINGS

FIG. 1 shows a side view of an exemplary embodiment of the present invention;

FIG. 2 shows a bottom plan view of the exemplary embodiment of FIG. 1;

FIG. 3 shows a close up sectional view taken across line 3-3 of the exemplary embodiment of FIG. 1;

FIG. 4 shows a top plan view of the exemplary embodiment of FIG. 1;

FIG. 5 shows a close up sectional view of another exemplary embodiment of the present invention;

FIG. 6 shows a top plan view of yet another exemplary embodiment of the present invention; and

FIG. 7 shows a top plan view of a still another exemplary embodiment of the present invention.

### DETAILED DESCRIPTION

The preferred embodiment and other embodiments, which can be used in industry and include the best mode now known of carrying out the invention, are hereby described in detail with reference to the drawings. Further embodiments, features and advantages will become apparent from the ensuing description, or may be learned without undue experimentation. The figures are not necessarily drawn to scale, except where otherwise indicated. The following description of embodiments, even if phrased in terms of “the invention” or what the embodiment “is,” is not to be taken in a limiting sense, but describes the manner and process of making and using the invention. The coverage of this patent will be described in the claims. The order in which steps are listed in the claims does not necessarily indicate that the steps must be performed in that order.

An embodiment of the present invention generally provides a bandage adapted to conform to contours of a portion of a human body. The bandage may comprise a conforming member forming one side of the bandage, such as a strip of flexible material with an inner side that adheres to the user. The conforming member may have two opposite ends and a central part. The two ends may have a pressure sensitive adhesive layer, such as an adhesive for attaching to human skin. Other embodiments may have other shapes, such as, for example, a circle with a raised central area.

Embodiments of a bandage may have a central part with a non-adhesive raised surface to pass air between a wound and the bandage when the bandage is applied to a user.

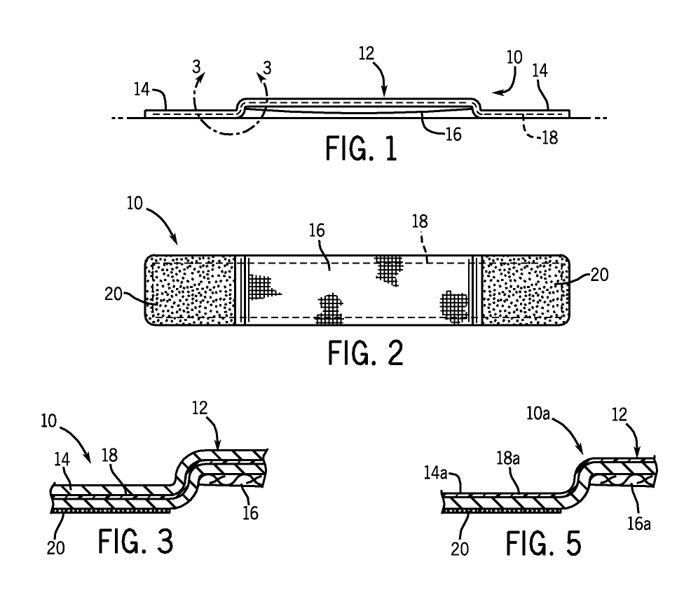

As depicted in FIG. 1, exemplary embodiments of the present invention may include a bandage 10 with a raised portion 12 and flat portions 14. The raised portion 12 may have gauze 16, so as to be non-adhesive, and the flat portions 14 may have an adhesive to adhere to a human body. A raiser 18 may be made of plastic and may lift a portion of the bandage 10 slightly by which it may allow air pass through between the wound and the bandage 10 and prevent the bandage 10 from sticking or rubbing the wound.

As depicted in FIG. 2, an embodiment of a bandage 10 may have a raiser 18 attached underneath or inside the bandage 10 and behind the gauze 16. The flat portions of the bandage 10 may have an adhesive 20, for releasably attaching the bandage to a user. In an embodiment, the raiser 18 may be attached to the bandage 10 utilizing the same adhesive 20 that adheres to the user.

As depicted in FIG. 3, an embodiment of a bandage 10 may have a built-in raiser 18 attached inside the bandage 10. The raiser 18 may have a rising portion that lifts the raised portion 12 above the flat portions 14, so that the gauze 16 is held above the layer of adhesive 20.

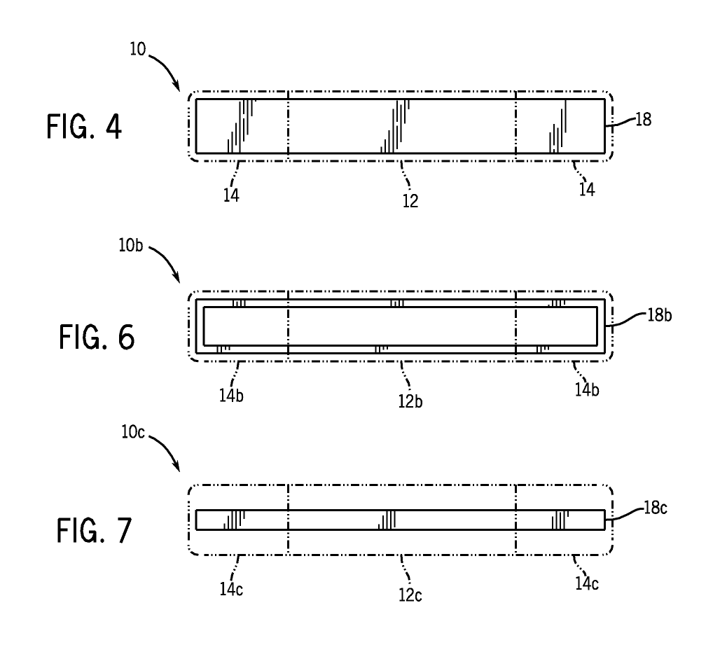

As depicted in FIG. 4, an embodiment of a bandage 10 may have a raiser 18 that forms surface covering a most of the raised portion 12 and flat portions 14. As depicted in FIGS. 4, 6, and 7, exemplary embodiments of a raiser 18 may be made in different sizes.

As depicted in FIG. 5, an embodiment of a bandage 10a may have a built-in raiser 18a attached outside the bandage 10a. The raiser 18a may be attached to the flat portions 14a opposite the adhesive 20, and may be attached to the raised portion 12 opposite the gauze 16a.

As depicted in FIG. 6, an embodiment of a bandage 10b may have a raiser 18b that forms loop near the edges of the raised portion 12b and flat portions 14b.

As depicted in FIG. 7, an embodiment of a bandage 10c may have a raiser 18c that is attached and disposed in the middle of the raised portion 12c and flat portions 14c, which only covers a portion of the bandage 10.

In an exemplary embodiment, the built-in raiser may have anti-bacterial properties and may come with different colors and sizes, for example.

An exemplary embodiment of the present invention may be designed and made by conventional manufacturing techniques in a conventional manufacturing facility. Users from all ages may be able to use the present invention whenever they want to use a bandage.

An embodiment of a bandage may comprise: a conforming member forming one side of the bandage, the conforming member having two opposite ends and a central part; a pressure sensitive adhesive layer on the two opposite ends; and a non-adhesive raised surface within the central part, the non-adhesive raised surface adapted to pass air between a wound and the bandage when the bandage is applied to a user.

I claim:

1. A bandage comprising:
a flexible material having a lower side;
first and second flat portions of the lower side of the material having an adhesive surface;
a raised portion of the lower side of the material having a non-adhesive surface; and
a raiser that transitions the material from the level of the first flat portion to the level of the raised portion and then back to the level of the second flat portion, so that the non-adhesive surface is raised relative to the adhesive surface.

2. The bandage of claim 1, wherein the non-adhesive surface is raised so as to pass air between a wound and the bandage when the bandage is applied to a user.

3. The bandage of claim 1, wherein the non-adhesive surface includes a layer of gauze.

4. The bandage of claim 1, wherein the first and second flat portions are opposite each other relative to the raised portion.

5. The bandage of claim 1, wherein the material is a strip of material that conforms to the human body, the first and second flat portions are ends of the strip, and the raised portion is the center of the strip.

6. The bandage of claim 1, wherein the material is a circle, the raised portion is a central raised area within the circle, and the flat portions are on opposite sides of the circle.

7. The bandage of claim 1, wherein the raiser is inside the flexible material.

8. The bandage of claim 1, wherein the raiser is on an outer side of the flexible material.

9. The bandage of claim 1, wherein the raiser is attached to the flexible material utilizing the adhesive surface.

10. A bandage comprising:
a conforming member forming one side of the bandage, the conforming member having two opposite ends and a central part;
a pressure sensitive adhesive layer on the two opposite ends; and
a non-adhesive raised surface within the central part, the non-adhesive raised surface adapted to pass air between a wound and the bandage when the bandage is applied to a user.

11. A raiser for a bandage having adhesive portions and a non-adhesive portion, the raiser comprising:
lower sections, for attaching to the adhesive portions of the bandage;
an upper section, for attaching to the non-adhesive portion of the bandage; and
transition sections that retain the upper section back from the lower sections so that the non-adhesive portion of the bandage is raised relative to the adhesive portions of the bandage.

12. The raiser of claim 11, wherein the non-adhesive portion of the bandage includes a layer of gauze, the adhesive portions of the bandage have a layer of adhesive, and the transition sections raise the gauze above the layer of adhesive so as to pass air between a wound and the bandage when the bandage is applied to a user.

13. The raiser of claim 11, wherein the raiser has anti-bacterial properties.
